(19) United States
(12) Patent Application Publication
(10) Pub. No.: US 2025/0284147 A1
(43) Pub. Date: Sep. 11, 2025

(54) NFC Tagged Eyeglasses

(71) Applicant: Stan Austin Soldner, Los Angeles, CA (US)

(72) Inventor: Stan Austin Soldner, Los Angeles, CA (US)

(21) Appl. No.: 19/075,041

(22) Filed: Mar. 10, 2025

Related U.S. Application Data
(60) Provisional application No. 63/563,742, filed on Mar. 11, 2024.

(51) Int. Cl. G02C 11/00; G02C 7/10; G06K 19/077

(52) U.S. Cl. CPC G02C 11/10; G06K 19/07762; G02C 7/104

(57) ABSTRACT

A pair of eyeglasses is disclosed, the eyeglasses comprising a near-field tag identifier that comprises authentication information about the eyeglasses, usage information regarding therapeutic eyeglasses, or links or content that supplements the usage of the therapeutic eyeglasses.

### CROSS-REFERENCE TO RELATED APPLICATIONS

The present application takes priority from Provisional App. No. 63/563,742, filed Mar. 11, 2024, which is incorporated herein by reference.

### BACKGROUND

There are many types of eyewear that serve many different purposes. A person may wear eyeglasses to correct visual defects, reading glasses to correct age-related presbyopia, sunglasses to protect their eyes from the sun, safety glasses to protect their eyes from physical damage, or various forms of therapeutic glasses to improve their mental or psychological functioning.

Some types of glasses provide color therapy to the wearer by providing colored lenses; different colors have different effects on the wearer's psychological state.

For any type of glasses, it is important to a user to be able to verify that the glasses are authentic and manufactured by the claimed manufacturer, to be able to determine various information regarding the glasses, or to be able to access various content or physical locations by using the glasses.

### LIST OF FIGURES

FIG. 1 shows an embodiment of the eyewear of the present invention.

FIG. 2 shows an embodiment of the eyewear of the present invention.

### SUMMARY OF THE INVENTION

An object of the present invention is to provide eyewear containing a near-field tag identifier.

Another object of the present invention is to provide therapeutic eyewear containing a tag encoding content that may supplement the therapy provided by the eyewear.

Another object of the present invention is to provide eyewear containing a tag that can be used to activate an NFT.

In summary, the present invention provides a pair of eyeglasses comprising lenses and a frame, and a near-field tag identifier embedded in the frame, wherein the near-field tag identifier is able to communicate with a computing device. The frame may comprise an EVA foam material and the near-field tag identifier can be inserted into a slit cut into the EVA foam material.

The lenses may be colored lenses for providing color therapy, regular corrective lenses, safety lenses, or any other eyeglass lenses.

The near-field tag identifier may comprise information relating to the prescription, identity of the user to whom the glasses are prescribed, safety rating, information relating to the hazards the eyeglasses are designed to avert (for safety glasses), or information relating to a non-fungible token.

In an embodiment, where the lenses are colored lenses for providing color therapy, the near-field tag identifier may comprise information relating to the color therapy performed using the eyeglasses. For example, it could comprise instructions for how to use the lenses for therapeutic effect, recommended duration of wearing, time of day for wearing, recommended frequency of wearing, information on the number of sessions performed, duration of the sessions the patient has had, and history of the patient's use of the eyeglasses.

In an embodiment, where the lenses are colored lenses for providing color therapy, the near-field tag identifier may comprise content relating to the color therapy, such as music, meditation audio, or video content. The video content may comprise modified colors that are the most aesthetically appealing while the user is wearing the eyeglasses, or elements that are only visible while the user is wearing the eyeglasses.

In an embodiment, the near-field tag identifier may be connected to a non-fungible token. The value of the non-fungible token may be connected to the stock value for the manufacturer of the eyeglasses.

In an embodiment, the near-field tag identifier may provide a rollable link that is only accessible by touching the eyeglasses.

In an embodiment, the near-field tag identifier may provide a user with access to a physical or online location, or to augmented-reality content. The augmented-reality content may only be visible while the user is wearing the glasses.

### DETAILED DESCRIPTION OF THE PREFERRED EMBODIMENT

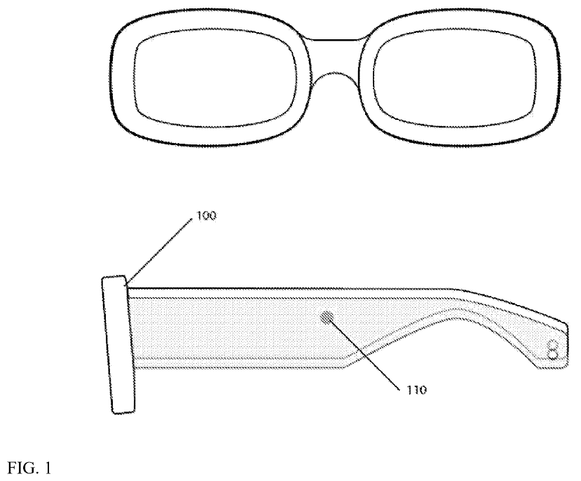

FIG. 1 shows an embodiment of the present invention. Eyeglasses 100 contain near-field tag identifier 110 that is permanently molded into the frame material. The near-field tag identifier is preferably molded into the frame material in such a way that it cannot be removed without destroying the frame.

In an embodiment, the frame is made out of acetate and the near-field tag identifier is embedded into the frame via a lamination process. In this embodiment, the frame is made out of two pieces and the near-field tag identifier is placed between them. The two pieces are then heat-pressed together or adhered together with acetone or other adhesive, with the near-field tag identifier between the two pieces.

The near-field tag identifier may be embedded into any part of the frame, as long as there is sufficient room for it. For example, it may be embedded into a temple, the tip of the temple, the nose bridge, or any other location where a near-field tag identifier can fit without compromising the strength of the frame.

In an embodiment, the frame of the eyeglasses 100 is made of EVA foam. This enables the glasses to float, which is important for outdoors applications such as boating or swimming. In this embodiment, the near-field tag identifier 110 may be embedded into the eyeglass frame as follows: a slit is cut into the material of the eyeglass frame, the near-field tag identifier 110 is embedded inside, and adhesive is used to close the slit.

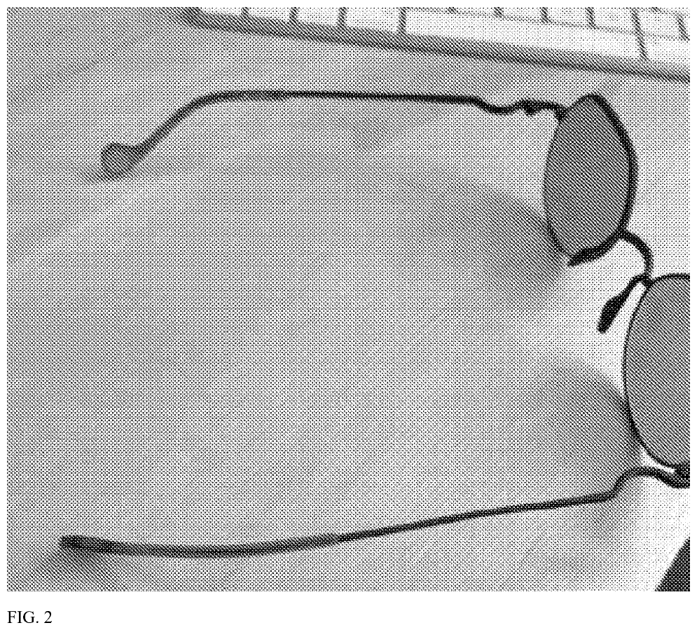

In an embodiment, the frame of the eyeglasses is made of metal. Since metal frames are thinner than plastic frames, the near-field tag identifier 110 may be embedded into the tip of a temple piece or attached to the tip of the temple piece, as shown in FIG. 2.

Near-field tag identifier 110 is preferably an NFC tag that is able to communicate with a mobile phone, a tablet, a computer, or any other computing device equipped with the capability of reading NFC tags. Near-field tag identifier 110 may also be another type of tag that may be read by a computing device at short range and does not require power, such as an RFID tag.

Near-field tag identifier 110 may contain any information, but preferably contains at least one or more of the following:
a. Identifying information for the eyeglasses, including manufacturer and serial number;
b. Information related to the eyeglasses, such as prescription, date of prescription, person for whom they are prescribed, lens type and rating (for safety glasses) and so on;
c. Address of at least one website containing content that supplements the color therapy;
d. At least some content that supplements the color therapy;
e. Information regarding an NFT.

The identifying information for the eyeglasses is presented in order to provide an easy way for a user to determine that the eyeglasses they have are authentic. For therapeutic purposes, it is especially important that lower-quality fakes should not be sold as the real thing, since the color therapy they provide may not be adequate. Similarly, for prescription glasses, quality is very important to prevent eyestrain. Even more importantly, for safety glasses, a poorly manufactured fake may risk the user's vision. For that purpose, the identifying information may include, but is not limited to: manufacturer name and serial number, date of manufacture, any parameters describing the lenses or frames, and any related information that would help the user determine that the eyeglasses are what they purport to be.

In an embodiment of the invention, the authentication may be performed by tapping a mobile phone to the glasses. The phone then reads the near-field tag identifier and determines the serial number of the eyeglasses. In an embodiment, the near-field tag identifier may also contain the ownership history of the eyeglasses.

For safety glasses, the near-field tag identifier may also contain information regarding the hazards the eyeglasses are designed to avert. For example, laser-safety glasses may be rated by the frequency they are designed to block; this information may be contained in the near-field tag identifier.

For color therapy glasses, the information related to the color therapy performed using the eyeglasses may be of two kinds. Firstly, this information may include instructions for how to use the eyeglasses for therapeutic effect, including but not limited to: recommended duration of wearing, time of day for wearing, recommended frequency of wearing, and other parameters that may be pre-programmed into the near-field tag identifier by the manufacturer or programmed into the near-field tag identifier by a medical professional. Secondly, the near-field tag identifier may also include information on the number of sessions performed, the duration of the sessions the patient has had, and other parameters describing the history of the patient's use of the eyeglasses.

The near-field tag identifier may also contain either content or links to online content to supplement the color therapy. In some cases, a user may want to listen to particular types of music while wearing the glasses (soothing music if the therapy centers on relaxation, or energizing music if the therapy centers on energy, or any other music to supplement the emotional effect provided by the glasses). A user may also want to follow along with meditation audio or video content while wearing the glasses, listen to ambient sounds, read or watch various content while wearing the glasses, and so on. The video content may have colors that are modified to be most aesthetically appealing while the user is wearing the glasses. In an aspect of the invention, the video content may have elements that are only visible while the user is wearing the glasses.

In an embodiment, the near-field tag identifier may provide a user with access to a physical or online location. For example, a user may use the near-field tag identifier to gain admittance to a music festival, or to log into an online forum.

In an embodiment, the link provided by the near-field tag identifier may be rollable; i.e. the online resource may only be accessed by touching the glasses, not by typing in a URL or forwarding the link to another person.

In an embodiment, the near-field tag identifier may provide access to augmented reality (AR) content. In an embodiment, the AR content may only be visible when the user is wearing the glasses. In an embodiment, the near-field tag identifier may activate a NFT (non-fungible token.) The NFT may contain a unit of “currency” based on the manufacturer's stock—i.e. the user may own part of the company by purchasing the glasses. The NFT may actually be connected to the manufacturer's stock shares and its value may depend on the stock value.

Exemplary embodiments are described above. It is understood that the present invention encompasses other embodiments that are perceived as reasonable equivalents to the abovedescribed elements by a person of reasonable skill in the art.

1. A pair of eyeglasses, comprising:
a frame;
two lenses installed in the frame;
a near-field tag identifier embedded in the frame;
wherein the near-field tag identifier is able to communicate with a computing device.

2. The pair of eyeglasses of claim 1, wherein the frame comprises an EVA foam material, and wherein the near-field tag identifier is inserted into a slit that is cut into the EVA foam material.

3. The pair of eyeglasses of claim 1, wherein the two lenses are colored lenses for providing color therapy.

4. The pair of eyeglasses of claim 1, wherein the near-field tag identifier contains at least one of the following information:
identifying information for the eyeglasses;
prescription information;
date of prescription;
person for whom the eyeglasses are prescribed;
safety rating and type of lens;
for safety glasses, information regarding the hazards the eyeglasses are designed to avert;
information regarding a non-fungible token.

5. The pair of eyeglasses of claim 3, wherein the near-field tag identifier comprises information relating to color therapy performed using the eyeglasses.

6. The pair of eyeglasses of claim 5, wherein the information comprises at least one of the following:
instructions for how to use the eyeglasses for therapeutic effect;
recommended duration of wearing;
time of day for wearing;
recommended frequency of wearing;
information on the number of sessions performed;
duration of the sessions the patient has had;
history of the patient's use of the eyeglasses.

7. The pair of eyeglasses of claim 5, wherein the information comprises content or links to online content to supplement the color therapy.

8. The pair of eyeglasses of claim 7, wherein the content comprises at least one of the following:
music;
meditation audio;
video content wherein the video comprises modified colors that are most aesthetically appealing while the user is wearing the glasses;
video content wherein the video comprises elements that are only visible while the user is wearing the glasses.

9. The pair of eyeglasses of claim 1, wherein the near-field tag identifier activates a non-fungible token.

10. The pair of eyeglasses of claim 9, wherein the value of the non-fungible token is connected to stock value for the manufacturer of the eyeglasses.

11. The pair of eyeglasses of claim 1, wherein the near-field tag identifier provides a rollable link, such that it can only be accessed by touching the eyeglasses.

12. The pair of eyeglasses of claim 1, wherein the near-field tag identifier provides a user with access to a physical or online location.

13. The pair of eyeglasses of claim 1, wherein the near-field tag identifier provides a user with access to an augmented-reality content.

14. The pair of eyeglasses of claim 13, wherein the augmented-reality content is only visible while the user is wearing the glasses.
